(19) United States
(12) Patent Application Publication
(10) Pub. No.: US 2012/0203058 A1
(43) Pub. Date: Aug. 9, 2012

(54) MOTION ACTIVATED ELECTRONIC THERAPEUTIC CUE DEVICE AND METHOD

(76) Inventors: Brian Keith Kanapkey, Chapel Hill, NC (US); Geri Tyndall Ronk, Durham, NC (US)

(21) Appl. No.: 13/023,592

(22) Filed: Feb. 9, 2011

(51) Int. Cl. A61M 21/00

(52) U.S. Cl. 600/26

(57) ABSTRACT

The present invention relates to a device and method for presenting audio, voice, and tone cues to aid in the rehabilitation of repetitive actions utilizing a device for actions such as eating, drinking or walking. The device also provides a diminisher function for varying the cues.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a device and method for presenting audio cues to a patient to improve carryover during rehabilitation of the patient. The present invention relates to a device and method for recording patient-specific audio cues which are motion activated during therapeutic activities such as walking, eating and drinking.

2. Description of Related Art

Dysphagia or swallowing problems can result from a number of situations and conditions, such as head and neck cancer, a stroke, brain tumors, brain injury, tracheotomy tubes, mechanical ventilation, and the like. An estimated 18 million or more Americans have difficulty with this otherwise routine function that, when not performed properly, can lead to aspiration pneumonia, malnutrition, dehydration, choking, and even death. Other conditions, such as walking, also require training to regain functional mobility.

Dysphagia rehabilitation involves training the patient to understand the components of swallowing and to make conscious efforts in the act of swallowing and repeating the act of swallowing multiple times. Much is involved with the specific act of swallowing, such as precise neuromuscular sequencing in movement, and voluntary and involuntary timing of each muscular activity as the patient swallows a liquid or solid. Since there is a wide variety of problems associated with dysphagia and each patient's musculature and swallowing needs are different, each patient presents a unique problem to the therapist teaching an individual patient to safely swallow.

Frequently, one or more verbal cues from the therapist is utilized to help remind the individual of the timing or physiologic action that the particular individual needs when relearning these activities. The cues act as a reminder or timing indicator for the activity. As the client improves (or in some cases, worsens) the verbal cues change to accommodate the individual patient's needs. The individual therapist or caregiver modifies the cues based on the individual patient's progress or lack thereof. However, few people can afford the time or money involved in having a therapist available for every needed activity for an extended period of time.

### BRIEF SUMMARY OF THE INVENTION

The present invention relates to a device and method for use by a patient/caregiver in a variety of rehabilitation settings. The invention comprises an electronic recording device attached to a therapeutic apparatus, such as an eating related utensil that can have specific audio cues (e.g. verbal or tone) recorded or played. The invention is motion activated, or otherwise triggered, during the activity to be cued.

Accordingly, one embodiment of the present invention relates to a device for delivering to a patient one or more customized audio cues during a therapeutic process utilizing an apparatus comprising:
a) an electronic audio playback and recording device having an on/off function for recording and playback of the audio cues;
b) a motion detector which senses motion of the apparatus during the therapeutic process and engages the playback of the recorded audio cues; and
c) a power source.

Yet another embodiment of the present invention relates to a method for delivering to a specific patient one or more audio cues during a therapeutic process utilizing an apparatus comprising:
a) selecting an electronic recording device having an on/off function comprising:
   i. an electronic audio playback and recording device having on/off function for recording and playback of the audio cues;
   ii. a motion detector which senses motion of the apparatus during the therapeutic process and engages the playback of the recorded audio cues; and
   iii. a power source;
b) recording one or more audio cues on the recording device when in record mode specific for the patient's needs during the therapeutic process;
c) positioning the recording device on or near the apparatus in playback mode sufficient that the motion detector can detect a desired motion of the apparatus; and
d) engaging the patient in the therapeutic process while the one or more cues are played back.

### BRIEF DESCRIPTION OF THE DRAWINGS

FIG. 1 is an embodiment of the present device on a drinking glass where tilting is detected.

FIG. 2 is an embodiment of the present invention on a fork where movement of the fork is detected.

FIG. 3 is an embodiment of the present invention on a plate for detection of a fork movement.

FIG. 4 is an embodiment of the present invention on a walker.

FIG. 5 is a flow chart of an embodiment of the present invention.

FIG. 6 is a block diagram of a device of the present invention.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the term “diminisher” refers to an adjuster device for an audio reminder with a tone. Over the course of repeating the therapeutic process (e.g. repeatedly drinking), the verbal reminder is removed or lessened in use (diminished) with only the tone remaining. Initially a tone is played every time the verbal cue is played. In diminished mode the tone is played every time and the verbal cue only played sometimes (e.g. every other time to every 5^{th} time).) Finally, the patient is reminded to perform each time with only the sound of the tone. As the patient progresses, the tones are also decreased (or diminished) until the patient has generalized or remembered the behavior without any verbal cues or tone cues.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “patient” refers to an individual person who is utilizing or has a need for therapeutic assistance with a repeated action utilizing an apparatus, such as repeatedly swallowing after using a fork to take a piece of food to eat. In general, any individual who is suffering from dysphagia or any condition to which rehabilitation therapeutic assistance is necessary, wherein audio, voice, or tone cueing is useful in aiding the person to learn a rehabilitation process or therapeutic strategy properly with repetitive actions. The audio verbal/tone cue creates memory response. In one embodiment, it increases the patient's swallowing safety, thus improving oral intake. Thus, audio cueing for this example refers to oral instructions of the steps involved, such as in the eating/drinking process including instructions for the process of eating such as “Position the food in your mouth” or the like or preparatory cues such as “Take smaller bites,” “don't forget to eat your high calories first,” “Take a drink after all bites of food,” and the like. These cues can relate to distinct verbal cues or be associated with selected tones.

As used herein, the “specific swallowing cue” or “customized audio cue” refers to the necessary audio verbal/tone cue or series of cues necessary for the particular patient to hear and remember to do the right things during the eating or other repetitive rehabilitation therapeutic processes involving an apparatus. In other words, a series of one or more (e.g. 2 or more) cues in a specific order and specific timing that meets the patient's specific needs for the patient's specific condition. As noted above, the number or type of cues may change to more or less cues, or longer or shorter cues as the individual patient gets better or worse during the therapeutic treatment. Each time the patient is about to take a drink, take a bite of food, take a step, etc., the audio specific cue(s) for the patient are activated by the motion detector. This provides the necessary reminders, training, and the like to repetitively perform the therapeutic activity safely. In another embodiment, the device is attached to a walker and activated as the walker is moved. It then provides an audio cue for the patient to ambulate or use the walker in a specific repetitive way as determined by the clinician or caregiver working with the patient that is specific to that patient.

As used herein, the term “therapeutic process” refers to the process which is restorative in nature (e.g. rehabilitation), is repetitive (e.g. patient does over and over, e.g. swallowing) and generally refers to regaining or improving a loss of function in the repetitive action (e.g. improve swallowing action). An “apparatus” refers to any object used in the repeated rehabilitation therapeutic process, such as an eating related utensil, walker, cane, weight, or the like.

As used herein, the “eating process” refers to the patient eating or drinking food or drink using an eating related utensil. An “eating related utensil” refers to the devices that accompany food and either hold or aid in the eating process. For example, cups, glasses, mugs, knives, forks, spoons, plates, bowls, placemats, and the like are all within the contemplated eating related utensil. In the case where a particular patient has other disabilities, the utensil may be an adapted utensil designed for a specific physical or mental handicap. Other devices used to hold or move food toward the mouth for the eating process are within the skill in the art.

As used herein the term “electronic audio playback and recording device” refers to a small lightweight digital type recorder or other small lightweight recording device that includes a playback device (speaker). The term “lightweight” means light enough to place on an apparatus if desired without causing problems with use, so less than 1 pound in one embodiment, in other embodiments less that 8 oz, 4 oz, 2 oz, or 1 oz. The recording device will be for recording one or more voice cues (in one embodiment) and its playback. Tones will automatically be associated with a voice cue. The device will have the ability for the user to record and re-record the speech for audio cues for the specific patient so the device must have a means of turning the recorder on and off for use and for recording and re-recording. In addition, it must have a button or the like for initiating the recording. Where tones are used, they can be turned on or off in one embodiment. As the cues are needed to be changed, the therapist or caregiver can delete and re-record the cues. The device must have a self-contained power source, like a battery or solar power as opposed to electric a/c type power with attached power cords.

The recorder can be made of any desired material for manufacturing a recording device, e.g. a digital recorder. In one embodiment, since the device may be exposed to food or drink, the device is made of water-resistant materials such as plastic, neoprene, rubber, silicone, or the like and sealed to be water proof (such as with gaskets and the like) if desired.

The recorder in one embodiment has an attachment device that allows the attaching of the recorder to the desired apparatus or object near the apparatus. For example, hook and loop fasteners, elastic bands, magnets, sticky material, and the like could be utilized to attach the recorder to the desired apparatus or apparatus or object.

As used herein, the phrase “motion detector” refers to a device in or attached to or with the recorder that senses a particular type of movement that will initiate the playback of desired audio verbal/tone cues. For example, where the device is on a cup or glass, the audio cues could be initiated with any movement of the cup or glass, the picking up of the glass or cup, the tilting of the cup or glass to a desired degree, for example, at least 20 or 30 degrees of tilt. In another embodiment, a free-floating switch is included on an eating utensil, such as a fork, and movement of the switch mechanism to a particular location initiates the series of cues. In yet another embodiment, the device attaches to one device, e.g. a plate, and comprises a sensor that can detect movement of another device, e.g. a hand or fork or the like, that is near the device, breaches a barrier such as the edge of the plate/placemat or the like and initiates the audio cues. In yet another embodiment, the device is attached to a walker, cane, or other therapeutic device and is used to provide audio cues to the patient, even in the absence of the therapist or caregiver.

In practicing the method of the present invention, the user turns on the recorder, making sure there is a battery or other functioning means to operate the recorder. In one embodiment, the desired verbal cues are recorded and tone cues are also utilized by associating the tone with a verbal cue. The tones are set so that preceding each cue, a tone sounds, followed by the recorded verbal cue. The tones are triggered by the movement of the device. While the tones will accompany each movement, the verbal cues can each be set to diminish, so that the tones will elicit the behavior until the behavior is generalized. The tones and cues can be set to diminish at a rate specified by the therapist using the diminisher function. For example, the tones can be set at 1:1 for a cue each time the trigger is activated, all the way to 1:5 where the verbal cue only plays once for every five tones played. Once recorded, the device is positioned on or near the therapeutic apparatus utilizing an attachment or the like. During the therapeutic process, the device is again turned on and the patient goes through the activity repeatedly where the device gives cues/tones when the proper apparatus motion is detected. Once the patient improves, cues/tones can be eliminated and/or changed as necessary with the ability to re-record verbal cues.

The manufacture of the present invention is within the skill of the art when given the present disclosure.

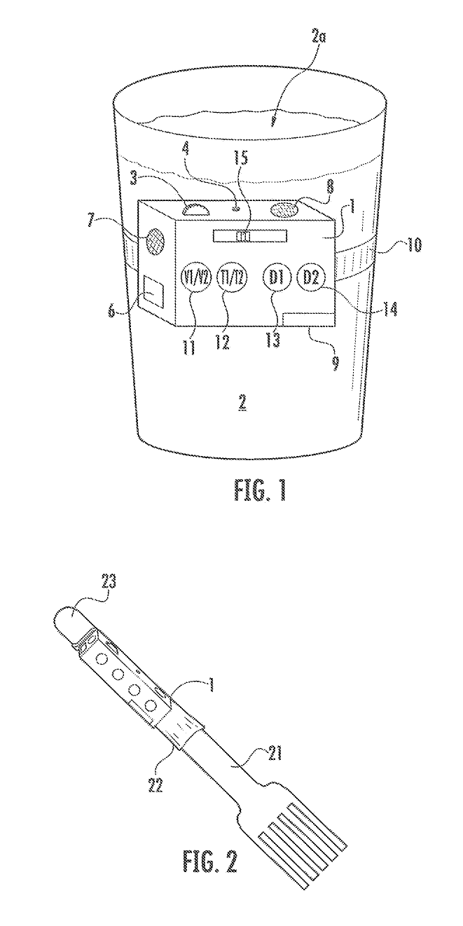

Now, referring to the drawings. FIG. 1 shows a device 1 of the present invention on a drinking glass 2 containing water 2a. The device 1 comprises an on/off switch and volume control 3 and a means for engaging the recording function 4. When recording the series of cues, this version of device 1 has the ability to select/record up to two voice cues. This is accomplished by holding the pen set 4 and hitting the V1/V2 button 11 one depression and record the voice cue into the microphone for up to 5 seconds to record for the first cue. For a second cue/voice, hold the pen set 4 and hit the V1/V2 button 11 two depressions and record the cue into the microphone for up to five seconds. To set the voice diminisher function, hold the pen set 4 and hit the D1 diminisher button 13 one time for constant voice cues in relationship to tone cues (1:1 ratio voice to tone) for every time the device is activated; hit D1 diminisher button 13 two times for voice cues every other time (1:2 voice cue every other tone cue) and hit the D1 button 13 five times for voice cues every fifth time the device is activated. To set the tone diminisher, hold the pen set 4 and hit D2, tone diminisher button 14 for constant tones (1:1 ratio) every time the device verbal cue is activated; hit two times for tones every other time (1:2) and hit the D2 button 14 five times for tones every fifth time the device is activated. To clear a previously set voice cue, hold the pen set 4 and T1/T2 button 12 for four seconds to clear the first voice recording; hold the pen set 4 and V1/V2 button 11 a second time for four seconds to clear the second voice recording. To clear previously set tones, hold pen set 4 and T1/T2 button 12 for four seconds to clear tone 1; hold pen set 4 and T1/T2 button 12 a second time for four seconds to clear tone 2. In other embodiments, the device has a pre-set demo mode which would have a pre-record voice option pre-set with similar voice cues. Another embodiment, of this invention would be a model which would only have the voice recording functions with no tone function. The tones involved are within the skill of the art in view of this disclosure in terms of selecting the sound, volume, and length of the tone. In another embodiment, the device could be equipped with a USB port/wireless technology port 6 to allow for a patient to receive cues directly to their ear to preserve dignity and/or help with those who have hearing loss use device effectively.

Shown on the device is recording microphone 7 and speaker 8 for listening to the series of cues. Also shown is sensitivity switch 15 for adjusting the sensitivity of the motion detector. The power source in this version is batteries which are placed in compartment 9 though other power sources and even solar power could be utilized.

The device 1 is attached to glass 2 via strap 10. In this embodiment, there is a tilt sensor inside the device 1. It is not seen but within the skill in the art to have a recorder that is activated by a tilt mechanism that detects a desired tilt to engage playing of the series of cues.

FIG. 2 shows the device 1 on a fork 21. It is attached to fork 21 by sleeve 22 which slips over the handle 23 of fork 21. Instead of a tilt sensor inside, this device has a motion detector and activates as the fork 21 moves in any manner. Function is as in FIG. 1.

FIG. 3 depicts one embodiment of the device 1, which comprises an on/off/volume switch 31 and a pen set 32 for engaging the recording function. Recording the series of cues in this version of the device is accomplished by depressing the pen set 32 and speaking into the microphone 33. The power source in this version is batteries which are placed in compartment 36, though other power sources and even solar power could be utilized.

The device 1 can be attached via an attachment device such as a strap 37 to the therapeutic apparatus of choice, e.g. plate 39a. For playback, press the playback button 34 with audio cue emitted from speaker 35. Motion detector 38 detects movement of an apparatus in this embodiment movement of fork 39 over plate 39a.

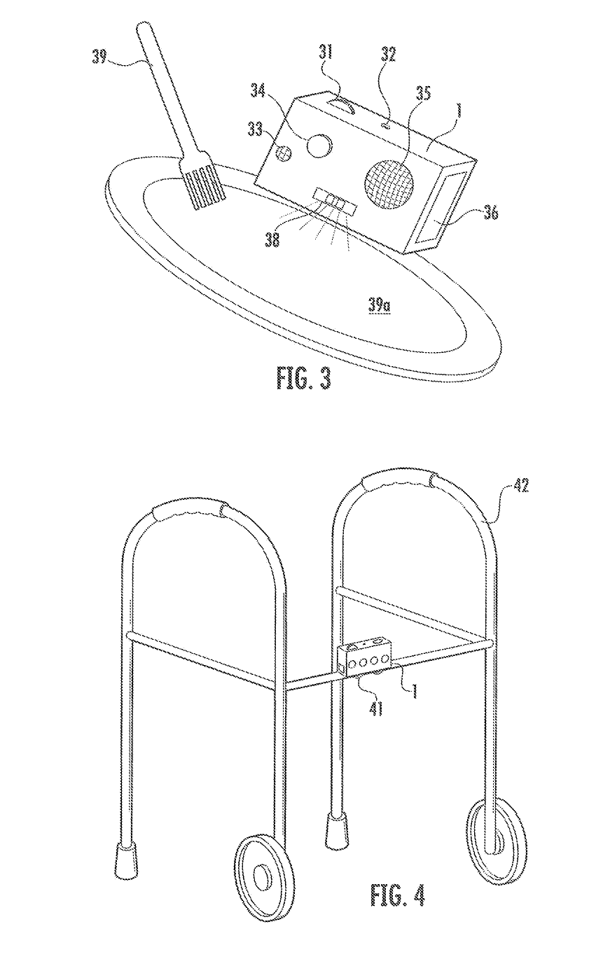

FIG. 4 depicts another embodiment of the invention 1 placed on a therapeutic device (a rolling walker). The device 1 is attached to the front of the walker 42 and attached with a strap 41 or the like. Once turned on and the walker is moved, the motion activated sensor is triggered and the audio cue (verbal cue and/or tone) sounds.

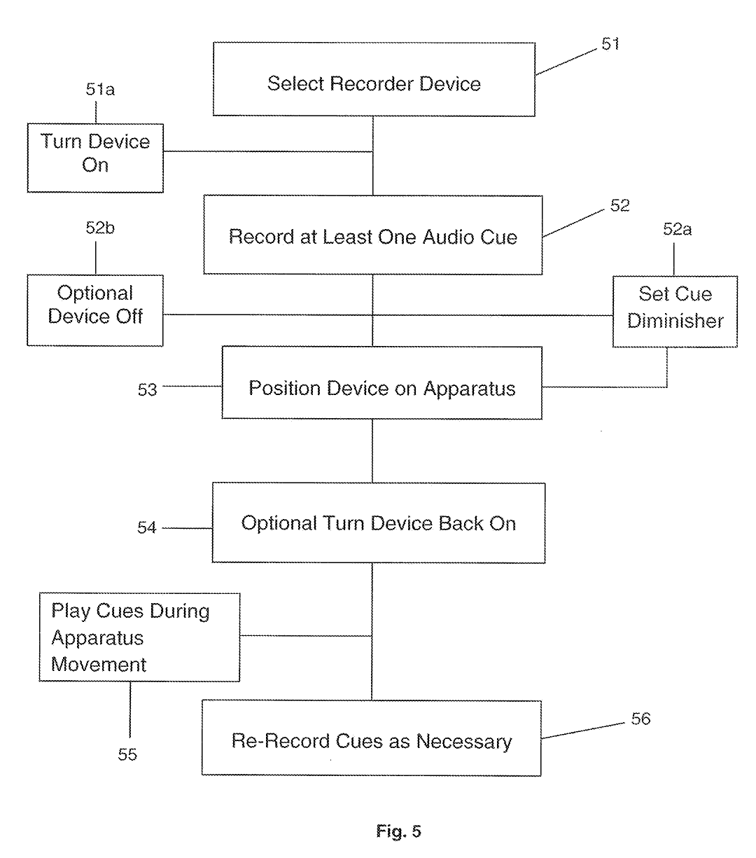

FIG. 5 is a flow chart that depicts a series of events or one embodiment of the method of the present invention. It should be noted that the events can be in altered order. The steps of the embodiment method are, first, a device as described above is selected 51. Once selected, the therapist, patient, or the like turns to device 51a and records at least one audio cue 52 for a selected apparatus. Where multiple apparatus are utilized, a series of cues for each utensil can be recorded. The cues and tone diminisher can be set 52a. The device can optionally be turned off 52b till it is ready to be used. The recorded device is positioned on an apparatus 53 for use in a therapeutic process. The device is then turned on during the therapeutic process 54 and the verbal/tone cues play as the patient performs the therapeutic function. The cues are then played during the apparatus movement 55. Optionally, where desired, the series of cues can be re-recorded 56 as needed to treat the changes in patient's progress or deterioration.

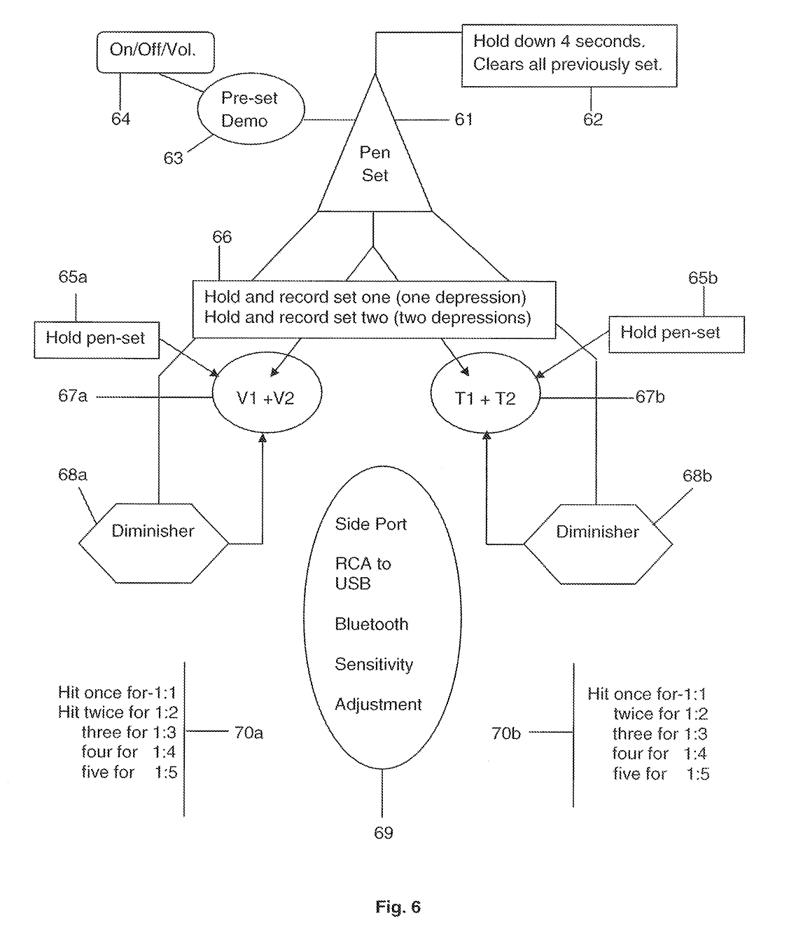

FIG. 6 is a block diagram of the diminisher utilized of the device embodied in FIG. 1. The pen set 61 initially can be used to initiate a preset demo 63 after the device is turned on by on/off/volume 64. If the device has prerecorded information on it, the pen set 61 can be held down for four seconds to clear an audio cue previously entered. The four seconds is an example and any desired time, number of presses, firmness of press, or the like that can be utilized to engage the pen set 61. The pen set 61 can be held down 65a and 65b to access the diminisher function. As noted then, pressing either diminisher 68a or 68b allows one to access the listed variations 70a and 70b of playing verbal or tone cues (or not played). For example, the 1:1 function would play one tone per one voice cue while, for example, the 1:5 function would play 5 tones first, then one voice cue as ratio suggests. The pen set can be held after one depression or two depressions 66 to access the record function of voice 67a or set/select tone 67b and record accordingly. The flow chart also notes that the side port has access to the Bluetooth function and sensitivity adjustment 69.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A device for delivering to a patient one or more customized audio cues during a therapeutic process utilizing an apparatus, comprising:
a) an electronic audio playback and recording device having on/off function for recording and playback of the audio cues;
b) a motion detector which senses motion of the apparatus during the therapeutic process and engages the playback of the recorded audio cues; and
c) a power source.

2. The device according to claim 1 wherein the device is positioned on the therapeutic device.

3. The device according to claim 2 wherein the device is positioned on a walker, knife, fork, or spoon.

4. The device according to claim 1 wherein the device is positioned on a plate.

5. The device according to claim 1 wherein the motion detected is a tilting of the apparatus.

6. The device according to claim 1 wherein the motion detected is the detection of a second apparatus in the proximity of a first apparatus.

7. The device according to claim 1 wherein the device further comprises a diminisher function.

8. The device according to claim 1 wherein the audio cue is selected from the list of voice cue; tone cue; and voice and tone cues.

9. The device according to claim 1 wherein the control of the device is by wireless remote.

10. The device according to claim 1 which further comprises an attachment device for attaching the recorder to the apparatus or other object.

11. The device according to claim 1 which further comprises a motion detector sensitivity control.

12. A method for delivering to a specific patient one or more audio cues during a therapeutic process utilizing an apparatus comprising:
a) selecting an electronic recording device having an on/off function comprising:
i. an electronic audio playback and recording device having on/off function for recording and playback of the audio cues;
ii. a motion detector which senses motion of the apparatus during the therapeutic process and engages the playback of the recorded audio cues; and
iii. a power source;
b) recording one or more audio cues on the recording device when in record mode specific for the patient's needs during the therapeutic process;
c) positioning the recording device on or near the apparatus in playback mode sufficient that the motion detector can detect a desired motion of the apparatus; and
d) engaging the patient in the therapeutic process while the one or more cues are played back.

13. The method according to claim 12 wherein the motion detected is by a tilting of the utensil.

14. The method according to claim 12 wherein the motion detected is by the detection of a second apparatus in the proximity of the first apparatus.
